(19) United States
(12) Patent Application Publication
(10) Pub. No.: US 2018/0057538 A1
(43) Pub. Date: Mar. 1, 2018

(54) POTENT AND HIGHLY SOLUBLE PEGYLATED COMPSTATIN PEPTIDES

(71) Applicant: The Regents of the University of California, Oakland, CA (US)

(72) Inventors: Dimitrios Morikis, Riverside, CA (US); Rohith R. Mohan, Riverside, CA (US); Reed E.S. Harrison, Riverside, CA (US); Ronald D. Gorham, Jr., Riverside, CA (US); Kaustabh Ghosh, Riverside, CA (US); Andrea P. Cabrera, Riverside, CA (US)

(21) Appl. No.: 15/685,364

(22) Filed: Aug. 24, 2017

Related U.S. Application Data
(60) Provisional application No. 62/379,907, filed on Aug. 26, 2016.

(51) Int. Cl. C07K 7/64; C07K 7/08; C07K 14/47; C07K 17/08; C08G 65/48

(52) U.S. Cl. CPC C07K 7/64; C07K 7/08; A61K 38/00; C07K 17/08; C08G 65/48; C07K 14/472

(57) ABSTRACT

The disclosure provides for highly soluble PEGylated compstatin peptides, which exhibit high binding affinities for complement and therapeutic efficacy in vitro. The disclosure further provides for pharmaceutical compositions comprising the PEGylated compstatin peptides, and methods of treatment thereof.

### CROSS REFERENCE TO RELATED APPLICATIONS

This application claims priority under 35 U.S.C. §119 from Provisional Application Ser. No. 62/379,907, filed Aug. 26, 2016, the disclosures of which are incorporated herein by reference.

### TECHNICAL FIELD

The disclosure provides for PEGylated compstatin peptides, pharmaceutical compositions comprising thereof, and methods of treatment therefrom.

### BACKGROUND

Many inflammatory and autoimmune diseases are initiated or mediated by dysregulation of the complement system, which is part of innate immunity and a link between innate and adaptive immunity. Currently, there are only two FDA approved complement therapeutics in the clinic and both of them are protein-based biopharmaceuticals. The approved complement therapeutics are equlizumab, and a human C₁-esterase inhibitor, which is based on a natural complement regulator protein. Although large biopharmaceuticals often confer high specificity for their targets, they suffer from poor stability, oral bioavailability and half-life, and are costly to produce.

### SUMMARY

The disclosure provides for highly soluble PEGylated compstatin peptides, which exhibit high binding affinities for complement and therapeutic efficacy in vitro. The disclosure further provides for pharmaceutical compositions comprising the PEGylated compstatin peptides, and methods of treatment therefrom. In a specific embodiment, the disclosure provides for a PEGylated compstatin peptide that combines an arginine-serine N-terminal polar amino acid extension and an 8-PEG block C-terminal extension. This PEGylated compstatin peptide demonstrated significantly improved aqueous solubility and efficacy in a human retinal pigmented epithelial (RPE) cell-based assay that mimics the pathobiology of age-related macular degeneration (AMD). In comparison to similar compstatin peptides, the PEGylated compstatin peptide had comparable inhibitory activity against complement activation, but exhibited improved efficacy in vitro. Moreover, the PEGylated compstatin peptide disclosed herein did not aggregate in solution. Accordingly, the PEGylated compstatin peptides of the disclosure are significantly better treatment options for disorders, like AMD, than other compstatin peptides which aggregate in solution.

In a particular embodiment, the disclosure provides for A PEGylated compstatin peptide consisting of a sequence selected from: X¹X²X³CVX⁴QDWGX⁵HRCT-[PEG]ₙ (SEQ ID NO:1), wherein, X¹ and X² may or may not be present, if X¹ and/or X² are present then X¹ and/or X² is independently selected from (a) any amino acid, (b) a polar group containing amino acid, or (c) S, W, meW, R, E, or N; X³ is W, MeW, R, I or L; X⁴ is W, meW, Nmw, V, Y or a non-natural amino acid analog of alanine (meW is a methylated tryptophan, Nmw is N-methyltryptophan; X⁵ is A or a non-natural amino acid analog of alanine; n is an integer from 4 to 20; and wherein the analog is acetylated at the N-terminus and amidated at the C-terminus, and wherein PEG is a small linear polyethylene glycol polymer, that acts as a solubilizer for the peptide. In another embodiment, X¹ is an R; X² is an S; X³ is an I; X⁴ is an W; X⁵ is an A; and/or n is an integer from 6 to 12. In a particular embodiment, the PEGylated compstatin peptide consists of the formula of Ac-RSICVWQDWGAHRCT-PEG₈-NH₂ (SEQ ID NO:2). In a further embodiment, the peptide is cyclized through a disulfide bond between the cysteine residues. In yet a further embodiment, the PEGylated compstatin peptide exhibits one or more of the following characteristics: having dissociation constant (K_{D}) for a complement protein C3c of less than 0.7 μM; reducing NHS-induced basal C5b-9n deposition in vitro by 80% or greater; and exhibiting high degree of solubility in aqueous solvents, with little to no visible or measured aggregation. In a certain embodiment, the PEGylated compstatin peptide has a dissociation constant (K_{D}) for a complement protein C3c of less than 0.7 μM, reduces NHS-induced basal C5b-9n deposition in vitro by 80% or greater, and is highly soluble in aqueous solvents, with little to no visible or measured aggregation. In a further embodiment, the complement protein C3c is human complement protein C3, C3b, and/or C3c. In yet a further embodiment, the PEG group has a high degree of local flexibility and global mobility that prevents self-association or aggregation of the PEGylated compstatin peptide.

In a particular embodiment, the disclosure also provides a pharmaceutical composition comprising a PEGylated compstatin peptide disclosed herein and a pharmaceutically acceptable diluent and/or excipient. In another embodiment, the pharmaceutical composition is formulated for intravitreal administration. In yet another embodiment, the pharmaceutical composition comprises a biodegradable polymer excipient.

In a certain embodiment the disclosure further provides a method to treat a complement mediated disease, disorder or condition in a subject in need thereof, comprising administering to the subject an effective amount of the pharmaceutical composition of the disclosure. In a further embodiment, the complement mediated disease, disorder or condition is selected from a group consisting of asthma, adult respiratory distress syndrome, hemolytic anemia, rheumatoid arthritis, rejection of xenotransplantation, stroke, heart attack, chronic obstructive pulmonary disease (COPD), paroxysmal nocturnal hemoglobinuria (PNH), atypical hemolytic-uremic syndrome (aHUS), dense deposit disease (DDD), C3 glomerulonephritis, and age-related macular degeneration (AMD). In yet a further embodiment, the complement mediated disease, disorder or condition is AMD.

### DESCRIPTION OF DRAWINGS

FIG. 1 shows concentration-dependent inhibition curves of compstatin peptides in four replicate hemolytic assay experiments. The plotted data represent the mean percent inhibition ±SEM. The dashed line intersects each inhibition curve at the IC₅₀. Peptide 1 is the parent peptide (positive control). Peptide 2 is the PEGylated form of the parent peptide. The third peptide is an unlabeled form of the Competition Peptide.

FIG. 2 demonstrates the solubility of compstatin peptides. Presented in the figure is the correlation of observed (measured) concentration with its calculated concentration. Peptide 2 was found to be the most soluble peptide, as is indicated by the high correlation between the calculated and observed concentrations (slope of 0.92). Each data point represents the mean measured concentration and the corresponding expected concentration with a linear regression fit to the data. A straight line of slope 1 passing through the origin is inserted to indicate closeness of the data to perfect correlation.

FIG. 3 displays the concentration dependent binding curve of Peptide 2 to C3c in competition with the Competition Peptide. The labeled form of the Competition Peptide was used. Thermophoretic data is plotted as mean±SEM from 7 replicate experiments, together with the fitted binding curve. The dotted lines in red indicate the 95% confidence interval of the fitted binding curve. The black dotted line represents the baseline thermophoretic response in the absence of binding. The inset table indicates the K_{D}±SEM.

FIG. 4 shows that Human RPE cells form basal deposits rich in C5b-9ₙ (MAC) in vitro, and shows the inhibitory effects of compstatin peptides. Representative cross-sectional images show three month-old RPE cell cultures labeled with anti-C5b-9ₙ (green) and DAPI (blue), following exposure to either 10% NHS alone or together with Peptides 1 or 2 for 24 h. Cultures that received no NHS treatment served as negative control. Scale bar: 10 μm.

FIG. 5 demonstrates that compstatin peptides inhibit complement-induced C5b-9ₙ deposition in RPE culture. Fluorescence intensity measurements from anti-C5b-9ₙ-labeled (decellularized) whole mount samples were normalized with respect to NHS-treated samples and plotted as mean±SEM. Data analyses show that Peptide 2 causes 80% reduction in NHS-induced basal C5b-9ₙ deposition by RPE cells. Peptide 1 exhibits weaker inhibitory effect, reducing C5b-9ₙ deposition by 50%. Bars indicate mean±SEM.

FIG. 6A-D provides the molecular structure of Peptide 2. (A) Representative conformers of Peptide 2 from the five highest-populated RMSD clusters of the molecular dynamics trajectory, with occupancies of 38%, 12%, 10%, 10%, and 9%. The peptide backbone is shown in a tube representation, colored by amino acid property type: gray for hydrophobic, green for polar, and brown for glycine. Cys2 and Cys12 are marked as hydrophobic because they form a disulfide bridge. The PEG₈ C-terminal extensions are shown in stick models of different colors for each conformer. The acetyl and amide terminal blocks are shown in stick models, colored by atom type: gray for carbon, white for hydrogen, blue for nitrogen, and red for oxygen. (B) The major conformer of Peptide 2 (38% occupancy) is presented in backbone tube representation, with key side chains of the compstatin peptide indicated in stick representation. The location of all amino acids is marked. The following color code is used: gray for hydrophobic, green for polar neutral, blue for basic, red for acidic, yellow for cysteines and disulfide bridge, and brown for glycine. The PEG₈-NH₂ C-terminal extension is shown in stick representation without hydrogens, with carbons depicted in brown, oxygens in red, and nitrogens in blue. (C) Surface representation of Peptide 2, with the PEG₈-NH₂ extension shown in stick representation (including hydrogens). The color code is as in Panel 4C, with the added hydrogens of PEG₈-NH₂ shown in white. (D) the chemical structure of PEG₈-NH₂.

FIG. 7 displays representative whole-mount images that show two month-old RPE cell cultures labeled with anti-C5b-9ₙ (green) following exposure to either 10% NHS alone or together with peptides 1 or 2 for 24 h. Cultures that received no NHS treatment served as negative control. Scale bar: 25 μm.

FIG. 8 displays representative whole-mount images that show ‘cell-free’ culture inserts labeled with anti-C5b-9ₙ (green) following exposure to 10% NHS for 24 h. No evidence of immunoreactivity is seen in the absence of RPE cells.

### DETAILED DESCRIPTION

As used herein and in the appended claims, the singular forms “a,” “an,” and “the” include plural referents unless the context clearly dictates otherwise. Thus, for example, reference to “a peptide” includes a plurality of such peptides and reference to “the pharmaceutical composition” includes reference to one or more pharmaceutical compositions and equivalents thereof known to those skilled in the art, and so forth.

Unless defined otherwise, all technical and scientific terms used herein have the same meaning as commonly understood to one of ordinary skill in the art to which this disclosure belongs. Although many methods and reagents similar or equivalent to those described herein can be used in the practice of the disclosed compositions and methods, provided herein are exemplary methods and materials.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

All publications mentioned herein are incorporated herein by reference in full for the purpose of describing and disclosing the methodologies, which might be used in connection with the description herein.

Inhibition of the complement system is a promising strategy to slow the progression of AMD pathogenesis. Currently, AMD is treated using monoclonal antibody based therapies targeting vascular endothelial growth factor (VEGF). VEGF stimulates choroidal neovascularization and induces vascular leakage. These therapies, however, are only shown to be effective for the wet (neovascular) form of AMD, which is associated with vessel rupture and local bleeding, but not in dry (atrophic) form of AMD, which is characterized by the accumulation of drusen deposits and RPE atrophy. By using phage display, functional, structural, and computational studies, a family of compstatin peptides were developed to inhibit complement-mediated autoimmune and inflammatory diseases. They, however, became attractive as low-molecular mass complement inhibitors for the treatment of AMD soon after a 2005 genomics study implicated complement involvement with AMD. The compstatin family peptides inhibit the cleavage of native C3 to its active fragments C3a and C3b. As a consequence, the deposition of C3b, the amplification of the alternative pathway and all downstream complement actions are prevented.

A “complement component” or “complement protein” is a molecule that is involved in activation of the complement system or participates in one or more complement-mediated activities. Components of the classical complement pathway include, e.g., C1q, C1r, C1s, C2, C3, C4, C5, C6, C7, C8, C9, and the C5b-9 complex, also referred to as the membrane attack complex (MAC) and active fragments or enzymatic cleavage products of any of the foregoing (e.g., C3a, C3b, iC3b, C3c, C3d, C4a, C4b, C5a, etc.). Components of the alternative pathway include, e.g., C3, C5, factors B, D, H, and I, and properdin.

The peptide compstatin binds to human and primate C3 and prevents its cleavage to C3a and C3b, a key step in complement activation. Compstatin also binds to the C3b fragment as well as the inactive C3c fragment, both of which contain the C3 β-chain. Compstatin has the sequence ICVVQDWGHHRCT (SEQ ID NO:3) and is maintained in a cyclic conformation via the disulfide bridge between the cysteines at position 2 and 12. Compstatin has been examined as a promising candidate for the treatment of unregulated complement activation.

One compstatin analog underwent clinical trials for AMD and, although it did not raise safety concerns, it did not show therapeutic efficacy. It is postulated that this failure was likely the results of molecular aggregation that resulted in the formation of gel-like structures and an associate loss of functionality. This analog had been optimized during several years to have a higher binding affinity than the original compstatin analogs by replacing a valine at position 4 with an aromatic amino acid, tyrosine or tryptophan, and subsequently with methylated tryptophan. The latter modification also increased the hydrophobic character of the peptide and presumably contributed to its aggregation in the aqueous ocular environment. Additional compstatin analogs are currently in clinical trials for a variety of complement-mediated diseases.

Recent studies have focused on increasing the solubility of compstatin peptides, using structure-based rational design, computational modeling, and optimization. These studies have identified several analogs with N-terminal extensions that have inhibitory activities similar to those of the most potent analogs, but have higher aqueous solubilities. Increased solubility was made possible by introducing two polar amino acid extensions at the N-terminus. In one analog an arginine at sequence position −1 not only contributed to solubility, but was also shown by molecular dynamics simulations to form a salt bridge with a glutamic acid in C3, thus contributing to binding affinity as well.

Using rational design methods, a number of PEGylated compstatin peptides were developed and are described herein. These PEGylated peptides were developed by employing design methods to identify, compstatin-based inhibitors of human C3.

As used herein, the term “PEGylated” or “PEGylation” in regards to a compstatin peptide, refers to a compstatin peptide that comprises or consists of one or more blocks of small linear PEG polymers. The one or more blocks of small linear PEG polymers are covalently attached to one or more of the amino acids making up the compstatin peptide. In a certain embodiment disclosed herein, one or more blocks of small linear PEG polymers are located at the C- end of the compstatin peptide, i.e., one or more blocks of small linear PEG polymers are covalently bound to C-terminal amino acid of the compstatin peptide. PEGylation of N-terminal amino acids likely will disrupt the N-terminal modifications of polar amino acids (when present) and/or sterically obstruct binding.

As used herein, the term “PEGylated compstatin peptide” refers to PEGylated peptides which exhibit complement inhibiting activity that is at least 50% as great as that of compstatin as measured, e.g., using any complement activation assay accepted in the art or substantially similar or equivalent assays. The assay may, for example, measure alternative pathway-mediated erythrocyte lysis or be an ELISA assay. The disclosure includes embodiments in which any one or more of the PEGylated compstatin peptides described herein is used in any the methods of treatment of a disease, disorder, or condition associate with aberrant complement activation. Examples of complement mediated diseases, disorders or conditions include, but are not limited to, inflammatory diseases, such as asthma, arthritis, and inflammatory bowel disease; autoimmune disorders, such as rheumatoid arthritis, systemic lupus erythematosus (SLE), neuromyelitis optica and antiphospholipid antibody syndrome (APS); ischemic diseases, such as stroke, and heart attack; transplant-related complications, such as rejection of xenotransplantation; lung diseases, such as chronic obstructive pulmonary disease (COPD), and chronic bronchitis; rare kidney diseases, such as paroxysmal nocturnal hemoglobinuria, membranous nephropathy, atypical hemolytic uremic syndrome, C3 glomerulonephritis and dense deposit disease (DDD); renal disease; glomerular disease; age-related and degenerative diseases such as age-related macular degeneration (AMD), and Alzheimer's disease; rare blood disorders, such as thrombotic thrombocytopenic purpura (TTP); and other disorders, such as acute respiratory distress syndrome, and hemolytic anemia.

The activity of a PEGylated compstatin peptide may be expressed in terms of its IC₅₀ (the concentration of the compound that inhibits complement activation by 50%), with a lower IC₅₀ indicating a higher activity as recognized in the art. The activity of a PEGylated compstatin peptide for use in the disclosure is at least as great as that of compstatin or substantially similar to that of compstatin. The IC₅₀ of compstatin has been measured as about 12.9 μM using an alternative pathway-mediated erythrocyte lysis assay. In one embodiment, the IC₅₀ of the PEGylated compstatin peptide is not more than the IC₅₀ of compstatin.

In certain embodiments of the disclosure the sequence of the compstatin PEGylated peptide comprises or consists essentially of or consists of a sequence that is obtained by making 1, 2, 3, 4, 5, 6 deletions, additions, substitutions and/or insertions in the sequence of compstatin (SEQ ID NO:3), e.g., 1, 2, 3, 4, 5, or 6 amino acids in the sequence of compstatin is replaced by a different standard amino acid or by a non-standard amino acid. In a certain embodiment of the disclosure, the amino acid at position 4 of SEQ ID NO:3 is altered. In another embodiment of the disclosure, the amino acid at position 9 of SEQ ID NO:3 is altered. In yet another embodiment of the disclosure, the amino acids at positions 4 and 9 of SEQ ID NO:3 are altered. In a further embodiment, a PEGylated compstatin peptide that comprises one or more amino acid replacements of the compstatin sequence, the PEGylated compstatin peptide may further comprise up to 1, 2, 3, or 4 additional amino acids at the N-terminus of SEQ ID NO:3.

In another embodiment, of the disclosure provides for a PEGylated compstatin peptide which comprises one or more naturally occurring amino acids and/or one or more non-naturally occurring amino acids. Examples of non-naturally occurring amino acids include singly and multiply halogenated (e.g., fluorinated) amino acids; D-amino acids; homo-amino acids; N-alkyl amino acids; dehydroamino acids; aromatic amino acids (other than phenylalanine, tyrosine and tryptophan); ortho-, meta- or para-aminobenzoic acid; phospho-amino acids; methoxylated amino acids; and α,α-disubstituted amino acids. In a specific embodiment, one or more non-naturally occurring amino acids are selected from 2-indanylglycine carboxylic acid (2IgI), dihydrotrpytophan (Dht), 4-benzoyl-L-phenylalanine (Bpa), 2-D-aminobutyric acid (2-Abu), 3-α-aminobutyric acid (3-Abu), 4-α-aminobutyric acid (4-Abu), 4-fluoro-L-tryptophan (4fW), 5-fluoro-L-tryptophan (5fW), 6-fluoro-L-tryptophan (6fW), 4-hydroxy-L-tryptophan (4OH-W), 5-hydroxy-L-tryptophan (5OH-W), 6-hydroxy-L-tryptophan (6OH-W), 1-methyl-L-tryptophan (IMeW), 4-methyl-L-tryptophan (4MeW), 5-methyl-L-tryptophan (5MeW), 7-aza-L-tryptophan (7aW), D-methyl-L-tryptophan (DMeW), β-methyl-L-tryptophan (βMeW), N-methyl-L-tryptophan (NMeW), Nmw (N-methyltryptophan), ornithine (orn), citrulline, norleucine, γ-glutamic acid, homocyclohexylalanine (hCha), N-methylalanine (Nma), R-α-ethylalanine (Rea), R-α-allylalanine (AaI), S-α-ethylalanine (Sea), β-(1-naphthyl)-L-alanine (1-Nal), β-(1-naphthyl)-D-alanine, β-(2-naphthyl)-L-alanine (2-Nal), β-(2-naphthyl)-D-alanine, β-(2-pyridyl)-L-alanine, β-(2-pyridyl)-D-alanine, β-(2-thienyl)-L-alanine, β-(2-thienyl)-D-alanine, β-(3-benzothienyl)-L-alanine, β-(3-benzothienyl)-D-alanine, β-(4-pyridyl)-D-alanine, β-(4-pyridyl)-D-alanine, β-chloro-L-alanine, β-cyano-L-alanine, β-cyclohexyl-L-alanine (Cha), β-cyclohexyl-D-alanine, β-cyclopent-1-yl-L-alanine, β-cyclopent-1-yl-D-alanine, β-cyclopentyl-L-alanine, β-cyclopentyl-D-alanine, β-t-butyl-L-alanine, and β-t-butyl-D-alanine.

In a particular embodiment, a PEGylated compstatin peptide disclosed herein comprises one or more analogs of Trp (e.g., at positions −1, 0, 4 and/or 7 relative to the sequence of compstatin of SEQ ID NO:3). Exemplary Trp analogs are mentioned above (e.g., methylated and halogenated Trp and other Trp and indole analogs). Other Trp analogs include variants that are substituted (e.g., by a methyl group). Amino acids comprising two or more aromatic rings, including substituted, unsubstituted, or alternatively substituted variants thereof, are of interest as Trp analogs.

In a certain embodiment, the disclosure provides for a PEGylated compstatin peptide that comprises one or more analogs of alanine (e.g., at position 9 relative to the sequence of compstatin (SEQ ID NO:3)), e.g., Ala analogs that are identical to Ala except that they include one or more CH₂ groups in the side chain. In a further embodiment, a PEGylated compstatin peptide comprises 2-Abu.

The disclosure provides for PEGylated compstatin peptides that consist of a sequence selected from: X¹X²X³CVX⁴QDWGX⁵HRCT-[PEG]ₙ, wherein X¹ and X² may or may not be present, if X¹ and/or X² are present X¹ and X² are independently selected from (a) any amino acid, (b) a polar group containing amino acid (e.g., K, R, H, D, E, C, Y, N, Q, S, T, W, A, G) or (c) S, W, meW, R, E or N; X³ is selected from W, meW, R, I or L; X⁴ is W or meW; X⁵ is A or a non-natural amino acid analog of alanine; and n is an integer selected from 2, 3, 4, 5, 6, 7, 8, 9, 10, 11, 12, 13, 14, 15, 16, 17, 18, 19, 20, 25, 30, 35, 40, 50, or a range between any two of the foregoing integers. In a further embodiment, X¹ is an R. In yet a further embodiment, X² is an S. In another embodiment, X³ is an I. In yet another embodiment, X⁴ is a W. In yet a further embodiment, X⁵ is an A. In a particular embodiment, n is either 6, 8, 10, or a range between 6 and 10. In still another embodiment, X¹ is an R, X² is an S, X³ is an I, X⁴ is a methylated tryptophan W and X⁵ is an A. In a particular embodiment of any of the foregoing, n is either 6, 8, 10, or a range between 6 and 10. In a certain embodiment, the disclosure provides for a PEGylated compstatin peptide that comprises or consists of a sequence as set forth above and which further comprises a N-terminal acetylation and/or a C-terminal amidation. In a particular embodiment, the disclosure provides for a PEGylated compstatin peptide which comprises, consists essentially of, or consists of the sequence of RSICVWQDWGAHRCT-PEG₈ or Ac-RSICVWQDWGAHRCT-PEG₈-NH₂ (both including SEQ ID NO:2).

As shown in the in vitro functional, binding, and solubility assays described herein, PEGylated compstatin peptides of the disclosure exhibit highly potent complement binding affinities, and are very soluble in aqueous solvents. The therapeutic efficacy of the PEGylated compstatin peptides of the disclosure to inhibit complement activation in a human retinal pigmented epithelial (RPE) cell-based assay is also shown herein. This RPE assay mimics AMD pathophysiology. Thus, the PEGylated compstatin peptides disclosed herein can be used as a therapy for treating dry and wet age-related macular degeneration (AMD) in a subject. Moreover, the PEGylated compstatin peptides of the disclosure demonstrate significantly improved aqueous solubility and complement inhibitory efficacy, compared to very similar peptides which lack PEG groups. Thus, the PEGylated compstatin peptides disclosed herein overcome the aggregation limitation for clinical translation that was shown with previous compstatin analogs. The PEGylated compstatin peptides of the disclosure are ideal candidates as a therapeutic treatment option for the treatment of AMD in a subject.

The PEGylated compstatin peptides of the disclosure may be prepared by various synthetic methods of peptide synthesis known in the art via condensation of amino acid residues, e.g., in accordance with conventional peptide synthesis methods, may be prepared by expression in vitro or in living cells from appropriate nucleic acid sequences encoding them using methods known in the art. For example, peptides may be synthesized using standard solid-phase methodologies. Potentially reactive moieties such as amino and carboxyl groups, reactive functional groups, etc., may be protected and subsequently deprotected using various protecting groups and methodologies known in the art. See, e.g., “Protective Groups in Organic Synthesis”, 3rd ed. Greene, T. W. and Wuts, P. G., Eds., John Wiley & Sons, New York: 1999. Peptides may be purified using standard approaches such as reversed-phase HPLC. Separation of diasteriomeric peptides, if desired, may be performed using known methods such as reversed-phase HPLC. Preparations may be lyophilized, if desired, and subsequently dissolved in a suitable solvent, e.g., water. The pH of the resulting solution may be adjusted, e.g. to physiological pH, using a base such as NaOH. Peptide preparations may be characterized by mass spectrometry if desired, e.g., to confirm mass and/or disulfide bond formation.

The natural or other chemical modifications such as those described above can occur anywhere in a peptide, including the peptide backbone, the amino acid side-chains and the N- and C-termini. A given peptide may contain many types of modifications. Peptides may be branched, for example, as a result of ubiquitination, and they may be cyclic, with or without branching. Peptides may be conjugated with, encapsulated by, or embedded within a polymer or polymeric matrix, dendrimer, nanoparticle, microparticle, liposome, or the like.

Peptides may, for example, be purified from natural sources, produced in vitro or in vivo in suitable expression systems using recombinant DNA technology in suitable expression systems (e.g., by recombinant host cells or in transgenic animals or plants), synthesized through chemical means such as conventional solid phase peptide synthesis and/or methods involving chemical ligation of synthesized peptides (see, e.g., Kent, S., JPeptScL, 9(9):574-93, 2003), or any combination of the foregoing. These methods are well known, and one of skill in the art will be able to select and implement an appropriate method for synthesizing the peptides described herein.

“Purified”, as used herein, means that the PEGylated compstatin peptides disclosed herein are separated from one or more other entities or substances with which it was previously found before being purified. The PEGylated compstatin peptides may be partially purified, substantially purified, or pure. The PEGylated compstatin peptide is considered pure when it is removed from substantially all other compounds or entities other than a solvent and any ions contained in the solvent, i.e., it constitutes at least about 90%, more typically at least about 91%, 92%, 93%, 94%, 95%, 96%, 97%, 98%, 99%, or greater than 99% of the dry weight of the composition. A partially or substantially purified PEGylated compstatin peptide may be collected from at least 50%, at least 60%, at least 70%, or at least 80% by weight of the material with which it is naturally found, e.g., cellular material such as cellular proteins and/or nucleic acids. In certain embodiments, a purified PEGylated compstatin peptide constitutes at least 10%, 20%, 30%, 40%, 50%, 60%, 70%, 80%, 90%, 95%, 99% or even more, by dry weight, in a composition. Methods for assessing purity are known in the art and include chromatographic methods, immunological methods, electrophoretic methods, etc.

The term “synthetic” or “non-natural” refers to a compound (e.g., a PEGylated compstatin peptide of the disclosure) that (i) is synthesized using a machine, or (ii) that is not derived from a cell or organism that normally produces the compound.

A “subject”, as used herein, refers to an individual to whom an agent is to be delivered, e.g., for experimental, diagnostic, and/or therapeutic purposes. Typically, subjects are mammals, particularly domesticated mammals (e.g., dogs, cats, etc.), non-human primates, or humans.

The PEGylated compstatin peptides of the disclosure may be formulated for delivery to a subject, may be conjugated to other compstatin analogs or conjugated to targeting molecules (e.g., linked to form, for example, a fusion construct), may be combined (e.g., two different compstatin agents) and/or may be concurrently administered with one or more additional therapeutic agents. The term “linked”, when used with respect to two or more moieties, means that the moieties are physically associated or connected with one another to form a molecular structure that is sufficiently stable so that the moieties remain associated under the conditions in which the linkage is formed and, typically, under the conditions in which the new molecular structure is used, e.g., physiological conditions. In certain embodiments of the disclosure the linkage is a covalent linkage. In other embodiments the linkage is noncovalent. Moieties may be linked either directly or indirectly. When two moieties are directly linked, they are either covalently bonded to one another or are in sufficiently close proximity such that intermolecular forces between the two moieties maintain their association. When two moieties are indirectly linked, they are each linked either covalently or noncovalently to a third moiety, which maintains the association between the two moieties. In general, when two moieties are referred to as being linked by a “linker” or “linking moiety” or “linking portion”, the linkage between the two linked moieties is indirect, and typically each of the linked moieties is covalently bonded to the linker. The linker can be any suitable moiety that reacts with the two moieties to be linked within a reasonable period of time, under conditions consistent with stability of the moieties (which may be protected as appropriate, depending upon the conditions), and in sufficient amount, to produce a reasonable yield. In some embodiments, the linker is a cleavable linker (e.g., a cleavable peptide).

In one embodiment, an appropriate binding moiety to which a PEGylated compstatin peptide is linked can be any molecule that specifically binds to a target molecule (e.g., polypeptide or a portion thereof). Such a binding moiety is referred to as a “ligand”. For example, in various embodiments of the disclosure a ligand can be a polypeptide, peptide, nucleic acid (e.g., DNA or RNA), carbohydrate, lipid or phospholipid, or small molecule (e.g., an organic compound, whether naturally-occurring or artificially created that has relatively low molecular weight and is not a protein, polypeptide, nucleic acid, or lipid, typically with a molecular weight of less than about 1500 g/mol and typically having multiple carbon-carbon bonds). Ligands may be naturally occurring or synthesized. Examples of ligands include, but are not limited to, hormones, growth factors, or neurotransmitters that bind to particular receptors.

“Concurrent administration” as used herein with respect to two or more agents, e.g., therapeutic agents, is administration performed using doses and time intervals such that the administered agents are present together within the body, or at a site of action in the body such as within the eye over a time interval in not less than de minimis quantities. The time interval can be minutes (e.g., at least 1 minute, 1-30 minutes, 30-60 minutes), hours (e.g., at least 1 hour, 1-2 hours, 2-6 hours, 6-12 hours, 12-24 hours), days (e.g., at least 1 day, 1-2 days, 2-4 days, 4-7 days, etc.), weeks (e.g., at least 1, 2, or 3 weeks), etc. Accordingly, the agents may, but need not be, administered together as part of a single composition. In addition, the agents may, but need not be, administered simultaneously (e.g., within less than 5 minutes, or within less than 1 minute) or within a short time of one another (e.g., less than 1 hour, less than 30 minutes, less than 10 minutes, approximately 5 minutes apart). According to various embodiments of the disclosure agents administered within such time intervals may be considered to be administered at substantially the same time. In certain embodiments of the disclosure concurrently administered agents are present at effective concentrations within the body (e.g., in the blood and/or at a site of action such as the retina) over the time interval. When administered concurrently, the effective concentration of each of the agents needed to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times. The de minimis concentration of an agent may be, for example, less than approximately 5% of the concentration that would be required to elicit a particular biological response, e.g., a desired biological response.

An “effective amount” of an active agent refers to the amount of the active agent sufficient to elicit a desired biological response. As will be appreciated by those of ordinary skill in this art, the absolute amount of a particular agent that is effective may vary depending on such factors as the desired biological endpoint, the agent to be delivered, the target tissue, etc. Those of ordinary skill in the art will further understand that an “effective amount” may be administered in a single dose, or may be achieved by administration of multiple doses. For example, an effective amount may be an amount sufficient to achieve one or more of the following: (i) inhibit or prevent drusen formation; (ii) cause a reduction in drusen number and/or size (drusen regression); (iii) cause a reduction in or prevent lipofuscin deposits; (iv) inhibit or prevent visual loss or slow the rate of visual loss; (v) inhibit choroidal neovascularization or slow the rate of choroidal neovascularization; (vi) cause a reduction in size and/or number of lesions characterized by choroidal neovascularization; (vii) inhibit choroidal neovascularization or slow the rate of retinal neovascularization; (viii) cause a reduction in size and/or number of lesions characterized by retinal neovascularization; (ix) improve visual acuity and/or contrast sensitivity; (x) inhibit or prevent photoreceptor or RPE cell atrophy or apoptosis, or reduce the rate of photoreceptor or RPE cell atrophy or apoptosis; (xi) inhibit or prevent progression of non-exudative macular degeneration to exudative macular degeneration; (xii) reduce one or more indicia of inflammation, e.g., the presence of inflammation-associated cells such as white blood cells (e.g., neutrophils, macrophages) in the eye, the presence of endogenous inflammatory mediators known in the art, one or more symptoms such as eye pain, redness, light sensitivity, blurred vision and floaters, etc.

A “macular degeneration related condition” refers to any of a number of disorders and conditions in which the macula degenerates or loses functional activity. The degeneration or loss of functional activity can arise as a result of, for example, cell death, decreased cell proliferation, loss of normal biological function, or a combination of the foregoing. Macular degeneration can lead to and/or manifest as alterations in the structural integrity of the cells and/or extracellular matrix of the macula, alteration in normal cellular and/or extracellular matrix architecture, and/or the loss of function of macular cells. The cells can be any cell type normally present in or near the macula including RPE cells, photoreceptors, and/or capillary endothelial cells. AMD is the major macular degeneration related condition, but a number of others are known including, but not limited to, Best macular dystrophy, Sorsby fundus dystrophy, Mallatia Leventinese and Doyne honeycomb retinal dystrophy.

“Local administration” or “local delivery”, in reference to delivery of a composition or agent of the disclosure, refers to delivery that does not rely upon transport of the composition or agent to its intended target tissue or site via the vascular system. The composition or agent may be delivered directly to its intended target tissue or site, or in the vicinity thereof, e.g., in close proximity to the intended target tissue or site. For example, the composition may be delivered by injection or implantation of the composition or agent or by injection or implantation of a device containing the composition or agent. Following local administration in the vicinity of a target tissue or site, the composition or agent, or one or more components thereof, may diffuse to the intended target tissue or site. It will be understood that once having been locally delivered a fraction of a therapeutic agent (typically only a minor fraction of the administered dose) may enter the vascular system and be transported to another location, including back to its intended target tissue or site.

Suitable preparations, e.g., substantially pure preparations of a PEGylated compstatin peptide disclosed herein, may be combined with pharmaceutically acceptable carriers, diluents, solvents, etc., to produce an appropriate pharmaceutical composition. The disclosure further provides a pharmaceutically acceptable composition comprising (i) a PEGylated compstatin peptide linked to a moiety (e.g., a ligand or cognate of a molecule on a cell surface) that binds to a component present on or at the surface of a cell or non-cellular molecular entity; and (ii) a pharmaceutically acceptable carrier or vehicle. The moiety may be an antibody or ligand. The component may be a marker such as a cell type specific marker for RPE or endothelial cells, a drusen constituent, and the like.

In certain embodiments, the disclosure provides a pharmaceutical composition comprising a PEGylated compstatin peptide disclosed herein which detectably inhibits development or progression of geographic atrophy and/or drusen formation in an eye, following administration to a subject. In other words, administration of the compound measurably reduces development or progression of geographic atrophy and/or drusen formation relative to the expected level in the absence of the composition. In another embodiment, the disclosure also provides a pharmaceutical composition comprising a PEGylated compstatin peptide disclosed herein which inhibits an increase in the retinal thickness (e.g., as measured by OCT) associated with a disease (e.g., the wet type of AMD). In yet another embodiment, the disclosure further provides a pharmaceutical composition comprising a PEGylated compstatin peptide disclosed herein which detectably inhibits vision loss in an eye, following administration to a subject. In other words, administration of the pharmaceutical composition measurably reduces vision loss relative to the expected level in the absence of the pharmaceutical composition. In a further embodiment, the disclosure provides a pharmaceutical composition comprising a PEGylated compstatin peptide disclosed herein which detectably inhibits inflammation in an eye, following administration to a subject. In other words, administration of the pharmaceutical composition which measurably reduces inflammation relative to the expected level in the absence of the pharmaceutical composition. It is to be understood that the pharmaceutical compositions of the disclosure, when administered to a subject, are preferably administered for a time and in an amount sufficient to treat or prevent the disease or condition for whose treatment or prevention they are administered. A useful pharmaceutical composition may provide one or more than one of the aforementioned beneficial effects.

The disclosure also provides for pharmaceutically acceptable compositions comprising a pharmaceutically acceptable derivative (e.g., a prodrug) of a PEGylated compstatin peptide disclosed herein, by which is meant any nontoxic salt, ester, salt of an ester or other derivative of a compound of the disclosure that, upon administration to a recipient, is capable of providing, either directly or indirectly, inhibition of the complement cascade.

An effective amount of the pharmaceutical composition is administered to a subject by any suitable route of administration including, but not limited to, intravenous, intramuscular, by inhalation, by catheter, intraocularly, orally, rectally, intradermally, by application to the skin, by eyedrops, etc. When a composition of the disclosure is used to treat an ophthalmic condition it will be appreciated that administration to the eye or in the vicinity of the eye will typically be used. A PEGylated compstatin peptide may be administered in a solid implant, or in a microparticle or nanoparticle formulation (e.g., a porous biocompatible and/or biodegradable nanoparticle), whereby it is protected from clearance and/or degradation in the bloodstream.

A pharmaceutical composition comprising a PEGylated compstatin peptide disclosed herein can be formulated for delivery by any available route including, but not limited to, parenteral, oral, by inhalation to the lungs, nasal, bronchial, ophthalmic, transdermal (topical), transmucosal, rectal, and vaginal routes. The term “parenteral” as used herein includes subcutaneous, intravenous, intramuscular, intra-articular, intra-synovial, intrasternal, intrathecal, intrahepatic, intralesional and intracranial injection or infusion techniques.

A pharmaceutically acceptable carrier or vehicle refers to a nontoxic carrier or vehicle that does not destroy the pharmacological activity of the compound with which it is formulated. Pharmaceutically acceptable carriers or vehicles that may be used in the compositions of this disclosure include, but are not limited to, water, physiological saline, and the like.

A pharmaceutical composition comprising a PEGylated compstatin peptide disclosed herein may include other components as appropriate for the formulation desired, e.g., buffer substances such as phosphates, glycine, sorbic acid, potassium sorbate, partial glyceride mixtures of saturated vegetable fatty acids, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Solvents, dispersion media, coatings, antibacterial and antifungal agents, isotonic and absorption delaying agents, and the like, compatible with pharmaceutical administration may be included. Supplementary active compounds, e.g., compounds independently active against the disease or clinical condition to be treated, or compounds that enhance activity of a compound, can also be incorporated into the compositions.

A pharmaceutically acceptable salt of a PEGylated compstatin peptide of the disclosure include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the disclosure and their pharmaceutically acceptable acid addition salts.

A pharmaceutical composition disclosed herein is formulated to be compatible with its intended route of administration. Solutions or suspensions used for parenteral (e.g., intravenous), intramuscular, intradermal, or subcutaneous application can include the following components: a sterile diluent such as water for injection, saline solution, dimethyl sulfoxide (DMSO), fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use typically include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.), phosphate buffered saline (PBS), or Ringer's solution.

Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In general, the pharmaceutical composition should be sterile, if possible, and should be fluid so that easy syringability exists.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Formulations for oral delivery may advantageously incorporate agents to improve stability within the gastrointestinal tract and/or to enhance absorption.

For administration by inhalation, the compositions are typically delivered in the form of an aerosol spray from a pressured container or dispenser which contains a suitable propellant, e.g., a gas such as carbon dioxide, or a nebulizer. Liquid or dry aerosol (e.g., dry powders, large porous particles, etc.) can be used. The disclosure also contemplates delivery of compositions using a nasal spray.

For topical applications, the pharmaceutically acceptable compositions may be formulated in a suitable ointment containing the active component suspended or dissolved in one or more carriers. Carriers for topical administration of the compstatin analogs include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

For local delivery to the eye, the pharmaceutically acceptable compositions may be formulated in isotonic, pH adjusted sterile saline or water, either with or without a preservative such as benzylalkonium chloride. Alternatively, for ophthalmic uses, the pharmaceutically acceptable compositions may be formulated in an ointment such as petrolatum or as eyedrops. Methods of local administration to the eye include, e.g., choroidal injection, transscleral injection or placing a scleral patch, selective arterial catheterization, eyedrops or eye ointments, intraocular administration including transretinal, subconjunctival bulbar, intravitreous injection, suprachoroidal injection, subtenon injection, scleral pocket and scleral cutdown injection, by osmotic pump, etc. The agent can also be alternatively administered intravascularly, such as intravenously (IV) or intraarterially. In choroidal injection and scleral patching, the clinician uses a local approach to the eye after initiation of appropriate anesthesia, including painkillers and ophthalmoplegics. A needle containing the therapeutic compound is directed into the subject's choroid or sclera and inserted under sterile conditions. When the needle is properly positioned the compound is injected into either or both of the choroid or sclera. When using either of these methods, the clinician can choose a sustained release or longer acting formulation. Thus, the procedure can be repeated only every several months or several years, depending on the subject's tolerance of the treatment and response.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The PEGylated compstatin peptides can also be prepared in the form of suppositories (e.g., with conventional suppository bases such as cocoa butter and other glycerides) or retention enemas for rectal delivery.

In addition to the agents described above, the PEGylated compstatin peptides can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polyethers, and polylactic acid. Methods for preparation of such formulations will be apparent to those skilled in the art. Certain of the materials can also be obtained commercially from Alza Corporation and Nova Pharmaceuticals, Inc. Liposomal suspensions can also be used as pharmaceutically acceptable carriers. These can be prepared according to methods known to those skilled in the art. Liposomes, including targeted liposomes (e.g., antibody targeted liposomes) and PEGylated liposomes can be used. One of ordinary skill in the art will appreciate that the materials and methods selected for preparation of a controlled release formulation, implant, etc., should be such as to retain activity of the compound. For example, it may be desirable to avoid excessive heating of polypeptides, which could lead to denaturation and loss of activity.

The following examples are intended to illustrate but not limit the disclosure. While they are typical of those that might be used, other procedures known to those skilled in the art may alternatively be used.

### Examples

Peptide Design:

Peptide design focused on the identification of inhibitors against rat C3 (rC3) (Tamamis et al., Chem. Biol. & Drug Design, 79:703-718, 2012; which is incorporated herein by reference for all purposes). In its general implementation setup, this protocol has a sequence selection stage and a sequence validation stage. The selection stage employs integer linear optimization to identify amino acid sequences, which correspond to global energy minima of a given (rigid or flexible) template fold. Several structures were used from a simulation of the rC3:W4A9 complex as a structural template. The validation stage computes the approximate binding affinities of these sequences to the target protein, without restricting the conformational space or the binding mode of the complex. For the purposes of the analysis the identification of compstatin analogs with a binding mode similar to the one in the hC3c:W4A9 complex was used. Sequences identified by the second de novo stage underwent detailed atomistic MD simulations in explicit water, which assess the structural behavior and stability of complexes involving the sequences from the selection stage.

Definition of Structural Templates.

The sequence-selection template consisted of a truncated rC3 protein model with residues 329-534 (the compstatin binding site in the hC3:W4A9 complex). This template was based on the simulation system of the rC3:W4A9 complex. To model flexibility, the template was placed into eight distinct conformations, taken at 1-ns intervals from a 7-ns molecular dynamics (MD) simulation of the rC3:W4A9 complex. All conformations were aligned along the structure of the hC3c:W4A9 complex (PDB code: 2QKI); after this alignment, the coordinates of the compstatin variant were extracted from the hC3c:W4A9 complex and combined with each of the rC3 conformations, to create eight distinct rC3:compstatin complexes.

Mutation Sets.

The next step in the selection stage species is to specify a list of possible amino acid types for each position in the designed sequence. This mutation set can be general (e.g., all twenty natural amino acids at every position), or restricted by knowledge-based considerations or other criteria, such as the solvent-accessible-surface-area (SASA). Two mutation sets were used in the present design. The first set allowed mutations at positions 1, 3 and 4 of compstatin. The amino acid types at each position were based upon the observed SASA of the corresponding W4A9 residue in the rC3c complex simulations. If a residue of the bound W4A9 was more than 50% exposed to solvent, only hydrophilic amino acids were allowed. If a position was less than 20% exposed, only hydrophobic amino acids were allowed; otherwise, all amino acids were allowed. Based on this criterion, position 1 was allowed to select from a set of hydrophilic amino acids (G, A, P, R, K, D, E, N, Q, H, S, T) and the native amino acid Ile, position 3 was allowed to select from a set of hydrophobic amino acids (A, C, G, V, I, L, M, F, Y, W, T), and position 4 was allowed to select from all amino acids. The small amino acids A, T and G were allowed in all positions. This led to a total of 2860 possible sequences. The second mutation set was based upon results from the first. Of the three positions allowed to mutate in the first set, position 3 showed the least variability, with W being the dominant amino acid (26% probability); other mutations in this position included F (21.4%), M (14.9%), Y (13.5%), I (7.2%), T (5.3%), C (4.1%), V (3.7%), L (3%), A (0.9%). Thus, the second mutation set fixed W at position 3 and allowed the other two positions (1 and 4) to mutate as before. This led to a total of 260 sequences.

Peptide Synthesis and Testing.

Compstatin peptides 1 and 2 are manufactured using standard peptide synthesizers using established protocols, or alternatively be contracted/purchased from commercial vendors for peptides (e.g., WuXi AppTec (Shanghai, China)). Peptide 2 has 8 PEG blocks attached at the backbone of the C-terminal amino acid. A Competition peptide for use in the thermophoresis experiments was synthesized by ELIM Biopharm (Hayward, Calif., USA) in two versions. One version was labeled with the cyanine fluorophore Cy5, which was attached at the side chain of the preceding lysine, and had sequence Ac-I[CVWQDWGAHRC]TAGK-(Cy5)-NH₂(SEQ ID NO:4). Another version was unlabeled and had sequence Ac-I[CVWQDWGAHRC]TAGK-NH₂ (SEQ ID NO:4). All peptides were cyclized by a disulfide bridge between the two cysteine amino acids, and they were acetylated at the N-terminus and amidated at the C-terminus. All peptides had >95% purity, as determined by HPLC and MS.

Hemolytic Assay:

Rabbit erythrocytes (Complement Technology, Inc, Tyler, Tex., USA) were washed with PBS and then resuspended in a veronal-buffered saline solution containing 5 mM MgCl₂ and 10 mM EGTA (VBS-MgEGTA). Two-fold serial dilutions of the compstatin analogs were performed in round-bottom 96-well plates and then further diluted in VBS-MgEGTA. Normal human serum (NHS; Complement Technology, Inc) diluted in VBS-MgEGTA was added to each well followed by incubation at ambient temperature for 15 minutes. Subsequently, 30 μL of rabbit erythrocytes at a concentration of 1.25×10⁸ cells/mL were added to each well. Positive controls for lysis consisted of: (1) erythrocytes in deionized water and (2) erythrocytes in NHS diluted with VBS-MgEGTA. Negative controls for lysis consisted of: (1) erythrocytes in VBS-MgEGTA and (2) erythrocytes in NHS diluted in VBS-EDTA. Next, plates were incubated at 37° C. for 20 minutes and then ice cold VBS containing 50 mM EDTA was added to each well to quench hemolytic reactions. Plates were centrifuged at 1000×g for 5 minutes and the supernatant was diluted 1:1 with deionized water in flat-bottom 96-well plates. Absorbance was measured spectrophotometrically at 405 nm to quantify lysis.

Apparent Solubility Measurements:

Compstatin analogs were dissolved in phosphate buffered saline (PBS) at pH 7.4 to concentrations of 10, 7.5, and 5 mg/mL. At each concentration point, the peptide solutions were vortexed for 30 seconds and then centrifuged at 13000×g for 5 minutes. The supernatant was collected and measured 5 times spectrophotometrically at 280 nm. Optical densities were converted into concentration according to Beer-Lambert law. An extinction coefficient of 11125 M⁻¹ cm⁻¹ was used for each compstatin analog since each peptide contains two tryptophan amino acids (tryptophan extinction coefficient being 5562.5 M⁻¹ cm⁻¹).

Microscale Thermophoresis Assay:

The binding affinity of Peptide 2 was evaluated in a competitive microscale thermophoresis assay, using a Monolith NT.115 instrument (NanoTemper Technologies GmbH, Munich, Germany). Competition was performed against the Competition Peptide, labeled with Cy5 for signal detection. Two different 1:1 serial dilution series of Peptide 2 were performed in MST buffer (50 mM Tris-HCl, 150 mM NaCl, 10 mM MgCl₂, 0.05% Tween 20). The first dilution series started with a final concentration of 333 μM and ended in a final concentration of 40.69 nM, while the second dilution series started with a final concentration of 166.67 μM and ended in a final concentration of 20.35 nM. To each dilution series of Peptide 2, purified C3c (Complement Technology) and the Competition Peptide (unlabeled form) were dissolved to final concentrations of 117 nM (Peptide 2) and 50 nM (Competition Peptide). The resulting mixture was incubated for 15 minutes in the dark at room temperature. Following the incubation, the samples were loaded into hydrophilic capillary tubes and the thermophoretic response of the fluorescently labeled marker was measured. Each dilution series was performed in at least triplicate and the results from both dilution series were combined for estimation of the IC₅₀ through nonlinear regression.

The fragment C3c was chosen for the thermophoresis assay because it binds compstatin, but it does not contain the thioester domain (TED). Further, it is the co-crystal structure of C3c with a bound compstatin analog (Ac-I[CVWQDWGAHRC]T-NH₂ (SEQ ID NO:5)). The bound compstatin analog of the crystal structure is the parent peptide of Peptide 1, with Peptide 1 having an Arg-Ser extension at sequence positions −1 and 0. The internal thioester bond of the TED undergoes spontaneous hydrolysis in C3, and the TED is highly mobile in C3 and C3b, possibly obscuring the protein's thermodiffusion properties in a direct (non-competitive) binding assay.

RPE Cell Culture:

The in vitro model of drusen biogenesis was used according to Johnson et al. (Proceedings of the National Academy of Sciences of the United States of America 2011; 108(45):18277-82) and used in assessing the efficacy of compstatin peptides. Briefly, human fetal RPE cells within the second passage were grown on laminin-coated porous inserts (Millipore, Billerica, Mass., USA) in Miller medium supplemented with 5% fetal bovine serum (FBS, HyClone, Logan, Utah, USA) at 37° C. and 5% CO₂ in a humidified incubator. After culturing for 2-3 months, cells were rinsed with PBS and treated with Miller medium containing either 5% FBS (negative control) or 10% NHS without or with the inhibitory peptides (2 μM). The choice of 2 μM dose for this assay was based on the intent to use double the IC₅₀ concentration, which was determined to be ˜1 μM. All inhibitory peptides were pre-mixed with NHS on a rocker at room temperature for 30 min and then warmed to 37° C. prior to addition to RPE cell cultures for 24 h. Next, the cells were rinsed with PBS, fixed in 4% paraformaldehyde (PFA, Electron Microscopy Sciences, Hatfield, Pa., USA) for 20 min, and stored in 0.4% PFA until use in immunofluorescence assays.

Immunofluorescence of Sub-RPE Deposits:

Basal deposits formed by the RPE cultures were visualized by immunofluorescence, as described in Johnson et al. Briefly, porous inserts were excised with a scalpel, cut into ˜4 mm² pieces, and rinsed several times with PBS. Next, one half of all RPE culture inserts were embedded in 10% (w/v) agarose (Fisher, Waltham, Mass., USA) and sectioned at 100 μm using a vibratome, while the other half was subjected to decellularization (to remove RPE monolayer) with 1% Triton X-100 and 0.4% (v/v) ammonium hydroxide. The sections and decellularized inserts were blocked with 5% donkey serum in PBS containing 0.5% bovine serum albumin and 0.1% Triton X-100 (overnight at 4° C.) and labeled with mouse anti-C5b-9 (AbCam, Cambridge, Mass., USA) (overnight at 4° C. or 2 h at ambient temperature), followed by labeling (2 h at RT) with Alexa Fluor 488-labeled anti-mouse IgG (Life Technologies, Carlsbad, Calif., USA). The immunolabeled decellularized inserts were directly mounted in Fluoromount (Sigma, St. Louis, Mo., USA) while the sections were counterstained with DAPI, a nuclear stain, prior to mounting.

Confocal Imaging and Analysis:

Immunolabeled RPE sections and decellularized culture inserts were imaged using a Leica SP5 confocal microscope. Single-plane images from multiple non-overlapping areas were acquired using a 63× objective, and C5b-9n (MAC) immunofluorescence signals were quantified using ImageJ (NIH), as described Gorham et al. (Experimental Eye Research 2013; 116:96-108). Briefly, the thresholding tool of ImageJ was used to analyze each color channel separately, with the upper threshold set to include C5b-9n-associated fluorescence and the lower threshold set to eliminate any background fluorescence. For statistical analysis, fluorescence intensity measurements of C5b-9n were averaged with respect to the number of RPE nuclei per image (for vibratome sections) or total image area (for decellularized whole-mount samples) and then expressed as a percentage of the corresponding fluorescence intensities from samples exposed to NHS in the absence of any inhibitory peptide (positive control). All data were obtained from multiple images (n≧9) and expressed as mean±SEM. Statistical significance was determined using analysis of variance (ANOVA), followed by Tukey's post-hoc analysis (Instat GraphPad Software Inc, La Jolla, Calif., USA). Results were considered significant if p<0.05.

Structural Modeling:

A structural model of the 8 linked PEG blocks was generated using MOLDRAW and then attached to a structural model of Peptide 1 using structure editing tools in Chimera, to generate a structural model of Peptide 2. The structural model of peptide 1 was derived from molecular dynamics simulations, based on the crystal structure of bound compstatin. CHARMM parameters and topologies were utilized and modified to incorporate the peptide-like bond between PEG₈ and Peptide 1. Modifications were rationally chosen based on existing amino acid parameters and topologies. Angles and dihedral angles that did not have a counterpart in existing CHARMM parameters and topologies were generated using SwissParam.

Molecular Dynamics Simulation:

An explicit-solvent molecular dynamics simulation was performed, for 90 ns, using as initial structure the modeled Peptide 2 structure. The peptide was solvated in a TIP3P water box with dimensions of 75×57×67 Å and charges were neutralized with sodium and chloride counterions at 150 mM. Following 25,000 steps of conjugate gradient energy minimization, the system was heated from 0 to 300 K in 62 ps with protein atoms constrained to post-minimization positions. Subsequently, the system was equilibrated through 5 stages for 50 ps per stage. Force constants of 41.83, 20.92, 8.368, and 4.184 kJ/mol/Å² were applied during the first 4 stages respectively to harmonically constrain all protein atoms to their post-minimization positions. During the fifth stage of equilibration, only the backbone atoms were harmonically constrained using a force constant of 4.184 kJ/mol/Å². Following equilibration, a production run was performed for 90 ns with periodic boundary conditions, SHAKE algorithm, 2 fs timesteps, Langevin pressure and temperature controls and particle-mesh Ewald electrostatics. The molecular dynamics trajectory (9000 frames) was clustered using the root-mean-square deviation (RMSD) of the backbone or alpha carbon atoms of Peptide 2, and a representative structure from the highest-populated clusters was identified and depicted for molecular graphics visualization.

Optimization of the Aqueous Solubility while Maintaining the Binding Affinity of Peptide 1.

Previous studies have focused on improving the solubility of peptides that underwent clinical studies for AMD, with the sequence Ac-I[CV(meW)QDWGAHRC]T-NH₂. This peptide, however, had high aggregation propensities in aqueous solutions, leading to its reduced solubility compared with other less potent compstatin analogs.

Peptide 1 was redesigned to add PEG block extensions at the C-terminus (Peptide 2; Table 1).

TABLE 1
PEPTIDE SEQUENCES.

| | | Molecular |
|---|---|---|
| Peptide | Sequence (SEQ ID NO: 2) | Mass |
| 1 | Ac-RSI[CVWQDWGAHRC]T-NH₂ | 1856 |
| 2 | Ac-RSI[CVWQDWGAHRC]T-PEG₈-NH₂ | 2279 |

Peptide 1 is a positive control with a two-polar amino acid N-terminal extension. Peptide 2 is a new design that contains an 8-PEG block backbone extension. Brackets denote disulfide bridge cyclization between the two cysteine amino acids. Ac: acetylation blocking group; NH₂: amidation blocking group.
The choice of the extension at the C-terminus of Peptide 2 was guided by the results of molecular dynamics simulations, which had shown that the C-terminus of compstatin points away from the C3-binding site towards the solvent.

Thus, it was reasoned that such extensions would not interfere with the binding interface between the compstatin analog and C3. Peptide 2 contains 8 PEG blocks attached at the peptide backbone in the C-terminus. The choice of PEG blocks was guided by earlier surface plasmon resonance (SPR) and ELISA studies, which had shown that PEGylated compstatin peptides had higher solubility compared to non-PEGylated peptides with the same sequence. The addition of a spacer of 8 PEG blocks to compstatin analogs was deemed necessary for the SPR binding experiments in order to increase the space between the peptides and attachment to the streptavidin sensor chip via lysine-biotin binding. This spacer aimed to increase the mobility of the peptides, enhance their accessibility to C3, and decrease non-specific interactions, thus emulating unbound ligand states as closely as possible within the experimental constraints. The inhibitory activities of peptides with the same sequences, but without the PEG blocks, were tested using ELISAs in the same study. PEGylation is an established procedure in drug design and delivery, as it has been shown to increase aqueous solubility and bioavailability. Current PEGylation technology for PEGylating drug products utilizes branched PEG polymers which are typically Y-shaped or comb shaped, resulting in massive PEGylation of the drug product. Massive drug PEGylation, advantages may include enhanced structural/chemical stability and circulation lifespan, owed to shielding effects, which also typically reduce drug immunogenicity, antigenicity, and toxicity. Massive PEGylation has also been used in a compstatin variant with modified backbone, but in that case a large (40 kDa) Y-shaped PEG structure was attached either at the N- or C-terminus.

In certain embodiments disclosed herein, the PEGylated peptide disclosed herein is PEGylated with small linear PEG-blocks on the C-terminus. In a further embodiment, the PEGylated peptide disclosed herein comprises a small (423 Da) linear 8-PEG block polymer on the C-terminus. PEGylated peptides of the disclosure which comprise small linear PEG blocks are at least 100 fold smaller than compstatin peptides which are massively PEGylated. Additionally, as shown herein, the small size and sequence location of the small linear PEG blocks do not introduce steric hindrance effects in accessing the binding site on C3 and alleviates structural interference with the specific inter-molecular pairwise amino acid interactions that are important for binding affinity. Finally, the non-PEGylated part of the peptide sequences described herein is different from the massively PEGylated compstatin peptides.

PEGylated Peptides Dose-Response Curves for Complement Binding.

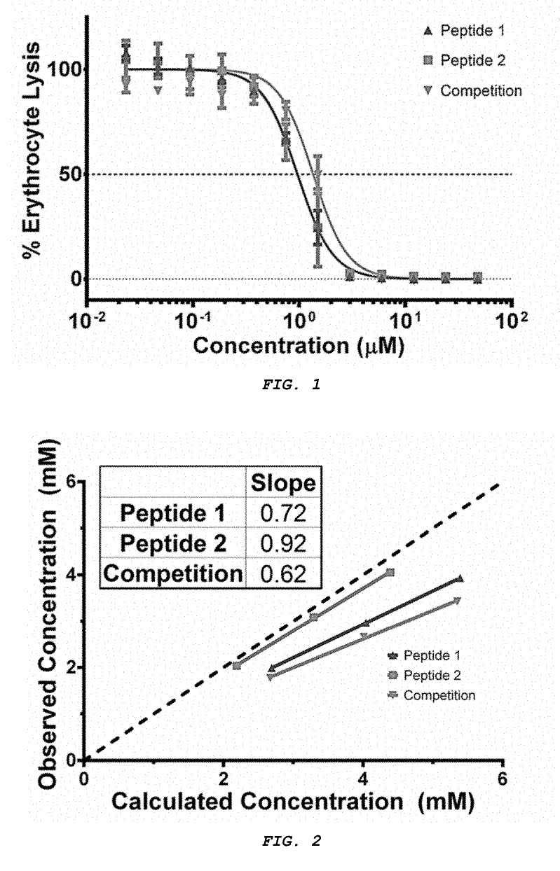

FIG. 1 displays dose-response curves of the complement hemolytic assay for PEGylated Peptide 2 and the parent Peptide 1 (positive control). Peptides 1 and 2 have similar IC₅₀ values within the confidence intervals from triplicate repeat experiments, and therefore similar potencies (Table 2). FIG. 1 also shows the dose-response curve of the unlabeled form of the Competition Peptide, which was used in a competitive binding experiment, vide infra.

Solubility of the PEGylated Peptides.

FIG. 2 shows a correlation plot between observed (experimental) and theoretical (calculated) peptide concentration in the range of 5-10 mg/mL. Concentration was experimentally measured using absorption spectroscopy at 280 nm, and calculated using the weight per volume values of the dilution series, as described herein. The data for Peptide 2 show the highest correlation among the three peptides, and its fitted straight line is closer to the straight line with slope 1 that passes through the origin (see FIG. 2). These data demonstrate that Peptide 2 has significantly higher apparent solubility than the parent Peptide 1 and the Competition Peptide.

Binding Affinities of the PEGylated Peptides.

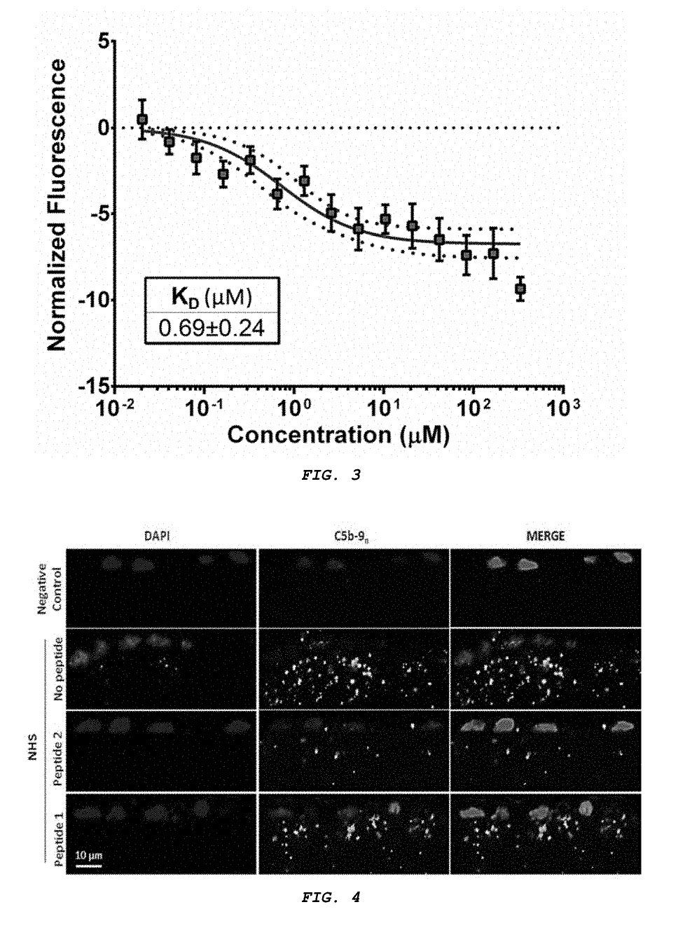

The binding affinity of Peptide 2 to C3c was determined using the microscale thermophoresis assay described herein. FIG. 3 displays the competitive binding of Peptide 2 to the C3c-Competition Peptide complex, where the Competition Peptide was labeled with the fluorophore Cy5 for detection of the thermophoresis signal. The unlabeled Competition Peptide has reduced potency compared to Peptide 2 (see Table 2 and FIG. 1), and for this reason was chosen for competitive replacement in the thermophoresis assay. This competitive binding experiment shows that Peptide 2 binds to C3c with a dissociation constant (K_{D}) of 0.69±0.24 μM.

TABLE 2
IC₅₀ VALUES FROM HEMOLYTIC ASSAY.

| | |
|---|---|
| | 95% |
| | Confidence |
| Mean IC₅₀ | Interval |

| Peptide | (μM) | Upper | Lower |
|---|---|---|---|
| 1 | 0.97 | 1.04 | 0.90 |
| 2 | 0.96 | 1.14 | 0.80 |
| Competition | 1.40 | 1.50 | 1.20 |

IC₅₀: Ligand concentration at 50% maximal inhibition. Data are from triplicate runs (N = 3). The Competition Peptide is unlabeled.

Efficacy of the PEGylated Peptides In Vitro.

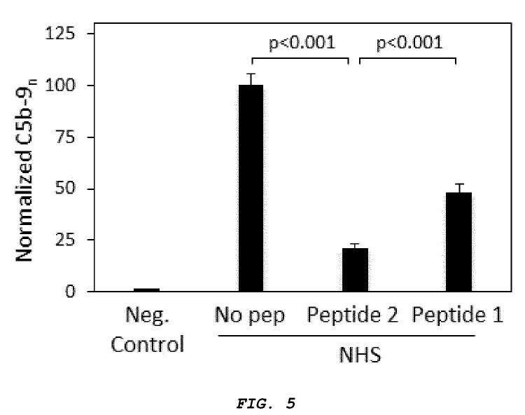

The efficacy of Peptides 1 and 2 were tested in a human RPE cell-based assay. This assay is based on a human RPE cell culture that mimics the pathophysiology of AMD, and can be used to interrogate the effects of complement activation in AMD pathogenesis. The assay is useful for quantification of the effects of inhibitors on complement activation mediated by sub-RPE drusen-like deposits, typical of those present in early AMD. FIG. 4 shows the deposition of deposit-associated C5b-9ₙ following addition of NHS as a complement source. Confocal imaging of immunolabeled RPE culture sections (see FIG. 4) and (decellularized) whole mounts (see FIG. 8) revealed that deposit-associated sub-RPE C5b-9ₙ deposition is inhibited by both compstatin peptides. More specifically, quantification of anti-C5b-9 immunofluorescence from whole mounts revealed that Peptide 2 causes a remarkable 80% inhibition (p<0.001) of complement activation (see FIG. 5). The inhibitory effect of Peptide 2 was significantly greater (p<0.001) than that achieved by Peptide 1, which reduced C5b-9ₙ deposition to a lesser degree (50%; p<0.001).

In combination: the hemolytic assay, apparent solubility, binding affinity, and human RPE cell-based assay data indicate that Peptide 2 is a more promising compstatin analog for further optimization and potential clinical translation, compared to Peptide 1. The parent Peptide 1 had emerged to be the best analog up to now in previous studies, in terms of solubility/affinity balance and efficacy of complement inhibition in the human RPE cell-based assay and references therein for earlier optimization studies).

Molecular Features of the PEGylated Peptides.

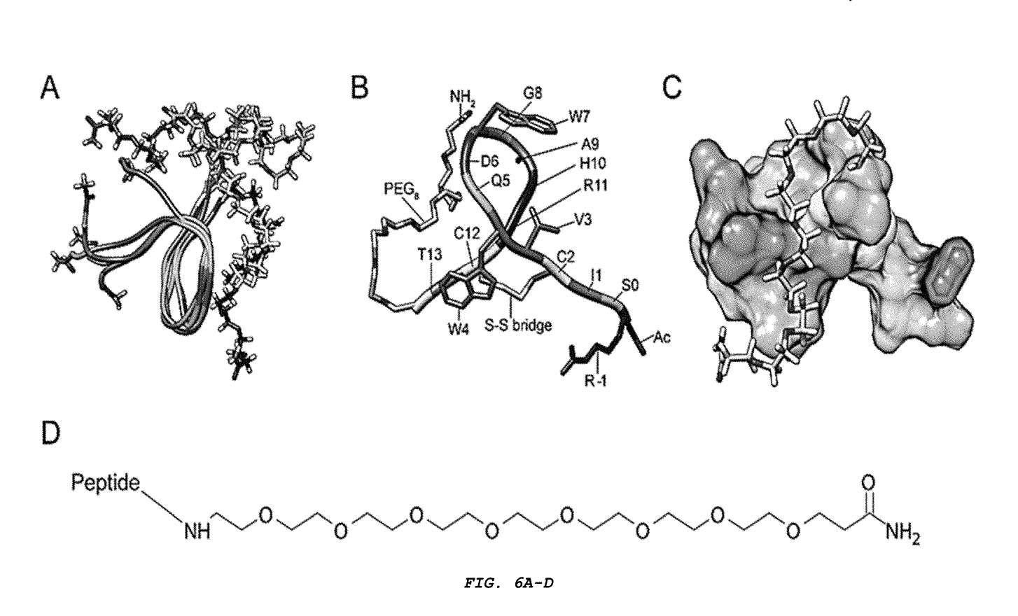

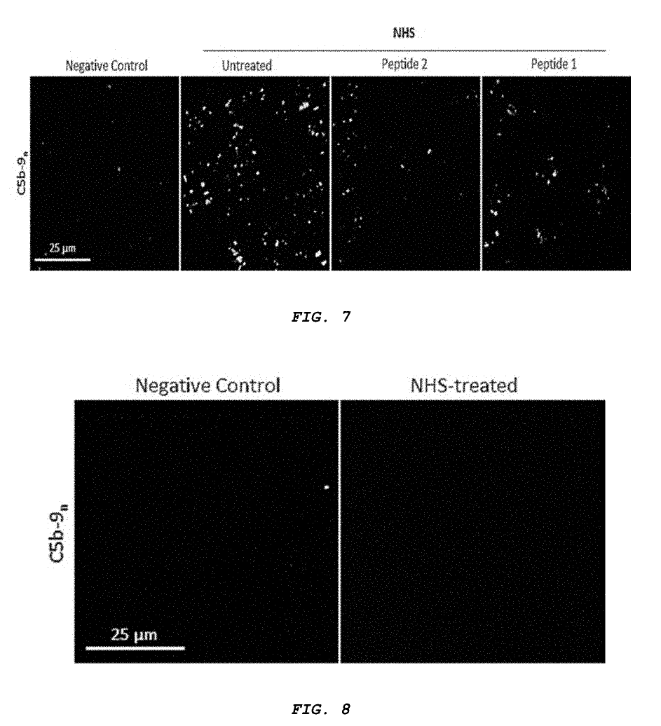

To gain insight into the molecular features that contribute to the structural stability and solubility of Peptide 2, an extended molecular dynamics simulation was performed. FIG. 6A shows representative conformations from the top (highest occupancy) five structural clusters derived from the molecular dynamics trajectory, using backbone atom RMSD-based clustering. These five clusters represent 79% of the conformations spanned by the peptide. The PEG₈ C-terminal extension demonstrates a high degree of local flexibility and global mobility, in essence forming a dynamic polar shell around Peptide 2. This dynamic polar shell perhaps functions as a shield from self-association and aggregation of Peptide 2 through hydrophobic features, thus contributing to the solubility of the peptide.

FIG. 6B shows the conformation of the representative Peptide from the structural cluster with highest occupancy, depicting key amino acid side chains for the optimization of compstatin in the past 20 years. These amino acids are Arg-1, Val3, Trp4, Trp7, Ala9, and the disulfide bridge Cys2-Cys12 (vide infra). The Arg-1 addition corresponds to the Arg-1/Ser0 extension of Peptide 1, the parent analog of Peptide 2. In this work is presented compstatin peptides were PEGylated with a PEG₈-NH₂ (see FIG. 6D) extension at the C-terminus (see FIG. 6A-C). They PEGylated compstatin peptides disclosed herein are the most promising compstatin analogs, in terms of affinity and solubility properties, up to now.

Effect of PEGylation on Compstatin Peptide Solubility and Binding Affinity.

Reported herein is the design of innovative compstatin peptides that have superior aqueous solubility and comparable binding affinity characteristics, compared to previously known peptides of the compstatin family. The peptide, Peptide 2 (see Table 1) is PEGylated and exhibits promising compstatin binding affinity and aqueous solubility (see Table 1). Peptide 2 has 8 PEG blocks attached to the backbone C-terminus, accounting for an additional molecular mass of 423 Da compared to Peptide 1 (see Table 1). Since the original discovery of compstatin using a phage-displayed random peptide library, there are many benchmarks in the optimization of the sequence of compstatin. Initial structure-inhibitory activity studies had derived a sequence template with 7 amino acids being indispensable for inhibitory activity and 6 amino acids being optimizable. FIG. 6B shows the side chains of amino acids for optimal binding and inhibitory activity of compstatin, including benchmark residue-specific optimization steps over the period of several years. Initial NMR, alanine scan, and inhibitory activity studies indicated that Val3 and Trp7 are important for binding to C3 and inhibition of complement activation. A subsequent crystal structure of C3c in complex with a compstatin analog confirm these findings, showing that Val3 and Trp7 are inserted in hydrophobic cavities. Benchmark optimization steps include the incorporation of: (i) Ala at position 9, which introduces helicity in the sequence and shifts a structural beta-turn from the central towards the C-terminal portion of the sequence; (ii) aromatic amino acids at position 4 with Trp being optimal, which was shown to participate in a hydrophobic clustering in the crystal structure; (iii) dipeptide N-terminal extensions, with Arg at position −1 being optimal because it increased solubility compared to previous peptides and also introduced a new inter-molecular salt bridge as shown by molecular dynamics simulations; and (iv) C-terminal extension using a 8-block PEG construct, which greatly increased aqueous solubility, which was unprecedented in comparison to other compstatin analogs. Also, acetylation at the N-terminus and amidation at the C-terminus contribute to improved activity. All active peptides contain a Cys2-Cys12 disulfide bridge.

Compstatin has two surfaces, a hydrophobic and a polar one, as it was pointed out by the original NMR studies, but it is the hydrophobic surface that makes the main contacts with the C3, as it was pointed out by the crystal structure. It is likely that the hydrophobic surface is responsible for the aggregation properties of the compstatin analog that underwent the early clinical trials. This analog contained a methylated Trp4 residue, in which the hydrophobic methyl group had replaced the polar hydrogen of the indole amide group, making the peptide even more hydrophobic. It was postulated that incorporation of polar amino acid extensions at the termini would increase solubility without perturbing binding properties, and this was shown to be the case for the N-terminus and C-terminus. However, the best aqueous solubility analog was found to be Peptide 2 that incorporates an 8-PEG block construct at the C-terminus. Molecular dynamic studies presented herein, demonstrate that the C-terminus is mobile pointing outwards from the C3-peptide interface towards the solvent. It was reasoned that the 8-PEG block construct will not sterically interfere with the binding interface of the C3-peptide complex. This is evident in FIG. 6, where the 8-PEG construct shows mobility around the non-binding site of Peptide 2, without specific contacts with the peptide. Therefore, the PEGylation acts as a solubilizer of compstatin.

Efficacy of the PEGylated Peptides In Vitro.

To determine their potential as AMD therapies, Peptide 2 was evaluated against its parent Peptide 1 in human RPE cell-based assays. The in vitro assay used herein is characterized by the formation of C5b-9-rich sub-RPE deposits, a hallmark of dry AMD. Thus, this assay represents a unique testbed for screening therapeutic candidates for treatment of dry AMD. This assay allows for the optimization of Compstatin peptide. For example, use of the assay demonstrated that incorporation of polar N-terminal extensions in the compstatin analogs. This assay has been used herein to demonstrate that Peptides 1 and 2 significantly inhibited the formation of C5b-9-rich sub-RPE deposits. Further, it was shown herein that PEGylated Peptide 2 exhibited a two-fold greater inhibitory effect than parent Peptide 1. This difference in anti-C5b-9 effect may be attributed to the greater solubility of Peptide 2, resulting in a higher “effective” concentration in the RPE culture.

A number of embodiments have been described herein. Nevertheless, it will be understood that various modifications may be made without departing from the spirit and scope of this disclosure. Accordingly, other embodiments are within the scope of the following claims.

What is claimed is:

1. A PEGylated compstatin peptide consisting of a sequence: (SEQ ID NO:1) X¹X²X³CVX⁴QDWGX⁵HRCT-[PEG]ₙ,
wherein,
X¹ and X² may or may not be present, if X¹ and/or X² are present then X¹ and/or X² is independently selected from (a) any amino acid, (b) a polar group containing amino acid, or (c) S, W, meW, R, E, or N;
X³ is selected from W, meW, R, I or L;
X⁴ is W, meW, Nmw, V, Y or a non-natural amino acid analog of alanine;
X⁵ is A or a non-natural amino acid analog of alanine;
n is an integer from 4 to 20; and
wherein the analog is acetylated at the N-terminus and amidated at the C-terminus, and wherein PEG is a small linear polyethylene glycol polymer that acts as a solubilizer for the peptide.

2. The PEGylated compstatin peptide of claim 1, wherein X¹ is an R.

3. The PEGylated compstatin peptide of claim 1, wherein X² is an S.

4. The PEGylated compstatin peptide of claim 1, wherein X³ is an I.

5. The PEGylated compstatin peptide of claim 3, wherein X³ is an I.

6. The PEGylated compstatin peptide of claim 1, wherein X⁴ is a W, meW or Nmw.

7. The PEGylated compstatin peptide of claim 5, wherein X⁴ is a W, meW or Nmw.

8. The PEGylated compstatin peptide of claim 1, wherein X⁵ is an A.

9. The PEGylated compstatin peptide of claim 7, wherein X⁵ is an A.

10. The PEGylated compstatin peptide of claim 1, wherein n is an integer from 6 to 12.

11. The PEGylated compstatin peptide of claim 1, wherein the PEGylated compstatin peptide has the sequence of SEQ ID NO:2 and the formula of Ac-RSICVWQDWGAHRCT-PEG₈-NH₂.

12. The PEGylated compstatin peptide of claim 1, wherein the peptide is cyclized through a disulfide bond between the cysteine residues.

13. The PEGylated compstatin peptide of claim 1, wherein the PEGylated compstatin peptide exhibits one or more of the following characteristics:
a dissociation constant (K_{D}) for a complement protein C3c of less than 0.7 μM;
reduces NHS-induced basal C5b-9n deposition in vitro by 80% or greater; and
exhibits high degree of solubility in aqueous solvents, with little to no visible or measured aggregation.

14. The PEGylated compstatin peptide of claim 13, wherein the PEGylated compstatin peptide has a dissociation constant (K_{D}) for a complement protein C3c of less than 0.7 μM, reduces NHS-induced basal C5b-9n deposition in vitro by 80% or greater, and is highly soluble in aqueous solvents, with little to no visible or measured aggregation.

15. The PEGylated compstatin peptide of claim 14, wherein the complement protein C3c is human complement protein C3, C3b, and/or C3c.

16. The PEGylated compstatin peptide of claim 1, wherein the PEG group has a high degree of local flexibility and global mobility that prevents self-association or aggregation of the PEGylated compstatin peptide.

17. A pharmaceutical composition comprising a PEGylated compstatin peptide of claim 1 and a pharmaceutically acceptable diluent and/or excipient.

18. The pharmaceutical composition of claim 17, wherein the pharmaceutical composition is formulated for intravitreal administration.

19. The pharmaceutical composition of claim 18, wherein the pharmaceutical composition comprises a biodegradable polymer excipient.

20. A method to treat a complement mediated disease, disorder or condition in a subject in need thereof, comprising administering to the subject an effective amount of the pharmaceutical composition of claim 14.

21. The method of claim 20, wherein the complement mediated disease, disorder or condition is selected from a group consisting of asthma, adult respiratory distress syndrome, hemolytic anemia, rheumatoid arthritis, rejection of xenotransplantation, stroke, heart attack, chronic obstructive pulmonary disease (COPD), paroxysmal nocturnal hemoglobinuria, atypical hemolytic uremic syndrome, dense deposit disease (DDD), C3 glomerulonephritis and age-related macular degeneration (AMD).

22. The method of claim 21, wherein the complement mediated disease, disorder or condition is AMD.


SEQUENCE LISTING (5 sequences)

SEQ ID NO: 1 - protein, 15 aa, Artificial Sequence
  Consensus peptide sequence
  MISC_FEATURE (1)..(2): Xaa at positions 1 and 2  may or may not be present, if Xaa at position 1 and/or Xaa at positions 2 are present earch are independently (a) any amino acid, (b) a polar amino acid (K, R, H, D, E, C, Y, N, Q, S, T, W, A, G) or (c) S, W, meW, R, E or N
  MISC_FEATURE (3)..(3): Xaa is W, meW, R, I or L
  MISC_FEATURE (6)..(6): Xaa is W or meW
  MISC_FEATURE (11)..(11): Xaa is 1-naphthylalanine or 2-naphthylalanine, Rea or Sea
  MISC_FEATURE (11)..(11): Xaa is a non-natural amino acid analog of alanine selected from 1-naphthylalanine or 2-naphthylalanine, Rea or Sea
XXXCVXQDWGXHRCT

SEQ ID NO: 2 - protein, 15 aa, Artificial Sequence
  Compstatin analog
RSICVWQDWGAHRCT

SEQ ID NO: 3 - protein, 13 aa, Homo sapiens
ICVVQDWGHHRCT

SEQ ID NO: 4 - protein, 16 aa, Artificial Sequence
  Compstatin Analog
ICVWQDWGAHRCTAGK

SEQ ID NO: 5 - protein, 13 aa, Artificial Sequence
  Compstatin analog
ICVWQDWGAHRCT